(19) United States
(12) Patent Application Publication
(10) Pub. No.: US 2013/0096692 A1
(43) Pub. Date: Apr. 18, 2013

(54) NON-CIRCULAR ESOPHAGEAL STENTS AND DELIVERY SYSTEMS

(71) Applicants: Mark D. Topazian, Rochester, MN (US); Louis-Michel Wong Kee Song, Rochester, MN (US); Todd H. Baron, SR., Oronoco, MN (US)

(72) Inventors: Mark D. Topazian, Rochester, MN (US); Louis-Michel Wong Kee Song, Rochester, MN (US); Todd H. Baron, SR., Oronoco, MN (US)

(73) Assignee: Mayo Foundation for Medical Education and Research, Rochester, MN (US)

(21) Appl. No.: 13/390,282

(22) PCT Filed: Aug. 12, 2010
(86) PCT No.: PCT/US2010/045293

§ 371 Date: Jun. 1, 2012

Related U.S. Application Data
(60) Provisional application No. 61/234,043, filed on Aug. 14, 2009.

(51) Int. Cl. A61F 2/82

(52) U.S. Cl. CPC A61F 2/82
USPC 623/23.7

(57) ABSTRACT

Stents that have non-circular cross-sectional profiles and systems for delivering the stents are described herein.

Esophageal stents that have a non-circular cross-sectional profile and delivery apparatus for deploying the esophageal stents are described herein.

Known esophageal stents with circular profiles can cause iatrogenic tracheo-esophageal fistula (TEF).

### SUMMARY

Stents that have non-circular cross-sectional profiles and systems for delivering the stents are described herein.

The stents may be useful in the proximal esophagus, where standard esophageal stents can cause stent-induced tracheo-esophageal fistula.

In some embodiments, the esophageal stents described herein may include a body member having a first end, a second end, and a length along a longitudinal axis extending between the first end and the second end; wherein the body member has a delivery configuration and a deployed configuration, wherein the body member is expandable from the delivery configuration to the deployed configuration; and wherein, in the deployed configuration, the stent has a non-circular cross-sectional profile in the absence of any external constraints acting on the stent, wherein the cross-sectional profile is determined in a plane transverse to the longitudinal axis, and wherein the cross-sectional profile defines a major axis extending across a maximum dimension of the cross-sectional profile and a minor axis transverse to the major axis at a midpoint of the major axis, wherein the body member is larger across the major axis than across the minor axis.

In some embodiments of the esophageal stents described herein, the non-circular cross-sectional profile may be an oval or elliptical cross-sectional profile.

In some embodiments of the esophageal stents described herein, the non-circular cross-sectional profile is substantially uniform along substantially the entire length of the body member.

In some embodiments of the esophageal stents described herein, the body member has a first major axis in a first cross-sectional profile obtained at a first location proximate the first end of the body member and a central major axis in a central cross-sectional profile obtained at a central location proximate a midpoint of the length of the body member, wherein the body member is larger across the first major axis than across the central major axis. The body member may have a first minor axis in the first cross-sectional profile and a central minor axis in the central cross-sectional profile, wherein the body member is larger across the first minor axis than across the central minor axis. The body member may have a first minor axis in the first cross-sectional profile and a central minor axis in the central cross-sectional profile, wherein the dimension of the body member across the first minor axis is substantially equivalent to dimension of the body member across the central minor axis.

In some embodiments of the esophageal stents described herein, the body member has a first major axis in a first cross-sectional profile obtained at a first location proximate the first end of the body member, a second major axis in a second cross-sectional profile obtained at a second location proximate the second end of the body member, and a central major axis in a central cross-sectional profile obtained at a central location proximate a midpoint of the length of the body member, wherein the body member is larger across the first major axis and the second major axis than across the central major axis. The dimension of the body member across the first major axis may be substantially equivalent to the dimension of the body member across the second major axis. In still other embodiments, the body member may have a first minor axis in the first cross-sectional profile and a central minor axis in the central cross-sectional profile, wherein the body member is larger across the first minor axis than across the central minor axis. In still other embodiments, the body member may have a second minor axis in the second cross-sectional profile, wherein the body member is larger across the second minor axis than across the central minor axis. In still other embodiments, the body member may have a first minor axis in the first cross-sectional profile and a central minor axis in the central cross-sectional profile, wherein the dimension of the body member across the first minor axis is substantially equivalent to the dimension of the body member across the second minor axis. In still further embodiments, the body member may have a second minor axis in the second cross-sectional profile, wherein the dimension of the body member across the second minor axis is substantially equivalent to the dimension of the body member across the central minor axis.

In some embodiments of the esophageal stents described herein, the stents may include an indicator on the body member, the indicator being indicative of the orientation of the rotational orientation of the body member about the longitudinal axis. The indicator may be a radiopaque indicator. The indicator may be a visual indicator visible to the unaided human eye.

In some embodiments, the stents described herein may include a tissue anchor located on an external surface of the body member. The tissue anchor may be a vertical tissue anchor that resists post-deployment movement of the stent in a direction along the longitudinal axis of the body member. The tissue anchor may be a rotational tissue anchor that resists post-deployment rotation of the stent about the longitudinal axis of the body member. In other embodiments, the tissue anchor resists post-deployment movement of the stent in a direction along the longitudinal axis of the body member and also resists post-deployment rotation of the stent about the longitudinal axis of the body member.

In some embodiments, the esophageal stent delivery systems described herein may include an esophageal stent that includes a body member having a first end, a second end, and a length along a longitudinal axis extending between the first end and the second end; wherein the body member has a delivery configuration and a deployed configuration, wherein the body member is expandable from the delivery configuration to the deployed configuration; and wherein, in the deployed configuration, the body member has an oval or elliptical cross-sectional profile in the absence of any external constraints acting on the stent, wherein the cross-sectional profile is determined in a plane transverse to the longitudinal axis, and wherein the cross-sectional profile defines a major axis extending across a maximum dimension of the cross-sectional profile and a minor axis transverse to the major axis at a midpoint of the major axis, wherein the body member is larger across the major axis than across the minor axis. The system also includes a delivery device restraining the body member in the delivery configuration.

In some embodiments of the stent delivery systems described herein, the body member is restrained within an interior of the delivery device.

In some embodiments of the stent delivery systems described herein the body member is restrained on an exterior of the delivery device.

In some embodiments of the stent delivery systems described herein, the delivery device includes an indicator that is indicative of the orientation of the rotational orientation of the body member of the stent about the longitudinal axis. The indicator may be a radiopaque indicator. The indicator may be a visual indicator visible to the unaided human eye. The visual indicator may be found in a shape of the delivery device.

In some embodiments, the methods described herein may include a method of supporting an esophagus that includes positioning a stent as described herein in an esophagus, wherein the positioning occurs while the body member of the stent is in the delivery configuration; and expanding the body member of the stent to the deployment configuration.

In some embodiments of the methods described herein, the positioning includes rotating the body member about the longitudinal axis before expanding the body member.

The above summary is not intended to describe each embodiment or every implementation of the present invention. Rather, a more complete understanding of the invention will become apparent and appreciated by reference to the following Description of Exemplary Embodiments and claims in view of the accompanying figures of the drawing.

### BRIEF DESCRIPTION OF THE VIEWS OF THE DRAWING

The present invention will be further described with reference to the figures of the drawing, wherein:

FIG. 1 is a perspective view of one embodiment of a stent as described herein.

FIG. 2 is a side view of another embodiment of a stent as described herein.

FIG. 3 is an end view of the stent of FIG. 2.

FIG. 3A is an end view of another embodiment of a stent that is flared in two directions.

FIG. 4 is a cross-sectional view of the stent of FIG. 2 taken along line 4-4 in FIG. 2.

FIG. 5 is profile view of another embodiment of a stent including directional indicators.

FIG. 6 is a view of the stent of FIG. 5 restrained within a delivery device.

FIG. 7 depicts an embodiment of a stent as described herein restrained on an exterior of a delivery device.

FIGS. 8A & 8B depict end and side views, respectively, of another embodiment of a delivery device.

FIG. 9 is a perspective view of another device that may be used in connection with the stents described herein.

### DETAILED DESCRIPTION OF EXEMPLARY EMBODIMENTS

In the following detailed description of illustrative embodiments, reference is made to the accompanying figures of the drawing which form a part hereof, and in which are shown, by way of illustration, specific embodiments of the stents and delivery systems described herein. It is to be understood that other embodiments may be utilized and structural changes may be made without departing from the scope of the present invention.

The stents described herein have non-circular profiles and are sized to maintain patency of the esophagus. The specific constructions used for the stents may vary considerably, e.g., the stents may be manufactured of any suitable material or combination of materials (e.g., metals, polymers, shape memory materials, etc.), the stents may be self-expanding or require an expansion force to expand them from a typically smaller delivery configuration into a typically larger deployment configuration, the stents may be have porous walls or walls that are impermeable to fluids and/or tissues; etc.

Such specific details of stent constructions are known and will not be described further herein, although reference may be had to the one or more of the following patents for further details regarding the construction, manufacturing, and/or deployment of stents: U.S. Pat. Nos. 4,733,665; 4,739,762; 5,195,984; 5,725,572; 5,735,871; 5,755,781; 5,853,419; 5,861,027; 6,007,573; 6,059,810; 6,099,561; 6,200,337; and 6,206,916; etc.

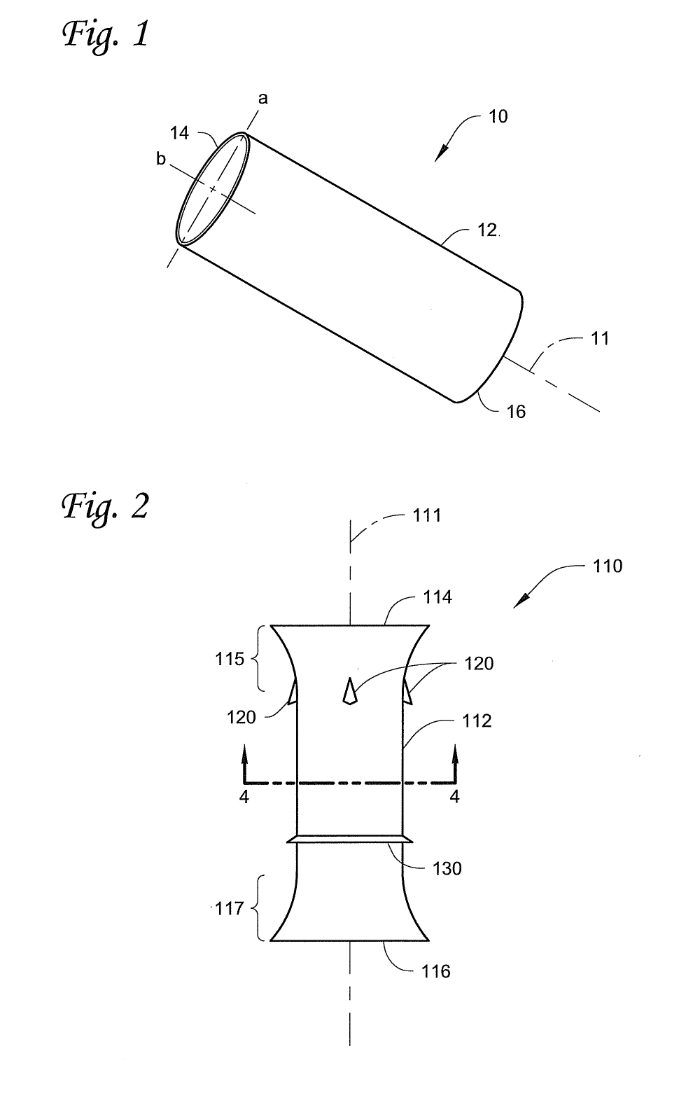

One illustrative embodiment of a stent as described herein is depicted in the perspective view of FIG. 1. The stent includes a body member 10 that has a wall 12 extending between a first end 14 and a second end 16. A longitudinal axis 11 extends between the first end 14 and the second end 16 such that the body member 10 has a length measured along the longitudinal axis between the first end 14 and the second end 16. The body member 10 has a delivery configuration and a deployed configuration, wherein the body member 10 is expandable from the delivery configuration to the deployed configuration. As seen in FIG. 1, the body member 10 is in the deployed configuration. When in the deployed configuration, the body member 10 defines a lumen through which fluids can pass from the first end 14 to the second end and vice versa.

Although the wall 12 of the body member 10 is depicted as solid, the wall could, in other embodiments, include apertures, openings, be constructed of a plurality of spaced-apart struts/wires, etc. as is known in the stent field. The construction of the non-circular stents described may vary about the perimeter of the stent to provide sufficient structural support to all portions of the esophagus about the entire perimeter of the stent. Variations may be provided in teens of thickness, materials, construction, etc. The variations may include use of materials, wires, struts, etc., of varying thickness and/or stiffness along different aspects of the stent perimeter and/or length, as well as, e.g., use of additional structural elements (such as additional wires, struts, etc.) along some aspects of the stent perimeter and/or length.

One feature that is depicted in the embodiment of FIG. 1 is the non-circular profile of the stent, where the profile is the shape of the stent taken in a cross-sectional plane oriented transverse to the longitudinal axis 11. In the embodiment of FIG. 1, the non-circular profile of the body member 10 may be described as an oval or elliptical cross-sectional profile. The body member 10 preferably takes a non-circular profile when in the deployed configuration and in the absence of any external constraints (other than, of course, gravity and ambient air pressure) acting on the stent. Those external constraints could include, e.g., a tube or delivery sheath into which the stent is inserted or restrained over.

In some embodiments, the stents described herein may, even in the absence of any external constraints, have a deployment profile (e.g., a circular profile, etc.) before deployment that is transformed to a non-circular profile during and/or after deployment. In some embodiments, such a stent may change profile due to a change in one or more different environmental characteristics such as, e.g., the presence of water or other aqueous fluids, changes in temperature, etc.

In some embodiments, the materials used to construct the stents may, for example, provide different expansile and/or containment forces after hydration which may occur as the stent is exposed to body tissues and/or fluids. The hydration may also be assisted by, e.g., providing a hydrating fluid after/upon deployment such as, e.g., saline, etc. In such embodiments, hydration of the stent materials may cause the stent to spontaneously transform from a deployment profile (e.g., a circular profile, etc.) to a non-circular profile as described herein.

In some embodiments, the materials used to construct the stents may, for example, provide different expansile and/or containment forces upon reaching body temperature after the stent is deployed in the selected internal body location. The change in temperature may, in some embodiments, be assisted by an external energy source that causes a temperature change in the stent materials. In such embodiments, temperature changes in the stent materials may cause the stent to spontaneously transform from a deployment profile (e.g., a circular profile, etc.) to a non-circular profile as described herein. Materials that are responsive to temperature changes and that could be used in the stents described herein may include, for example shape memory materials such as, e.g., nickel titanium alloys (e.g., Nitinol), etc.

The non-circular profile of the body member 10 may be described as being an oval or elliptical cross-sectional profile, where the term “oval or elliptical” includes shapes that are not true ovals or true ellipses, but are, instead, flattened curvilinear shapes (although true ovals and ellipses are included). More generally, the non-circular cross-sectional profiles may be described as defining a major axis (a) extending across a maximum dimension of the cross-sectional profile and a minor axis (b) transverse to the major axis at a midpoint of the major axis. The body member 10 is larger across the major axis than across the minor axis when the body member 10 is in the deployed configuration in the absence of any external constraints as described herein.

Another embodiment of a stent is depicted in FIG. 2 and includes a body member 110 having a wall 112, a first end 114 and a second end 116. The body member 110 further defines a longitudinal axis 111 extending between the first end 114 and the second end 116.

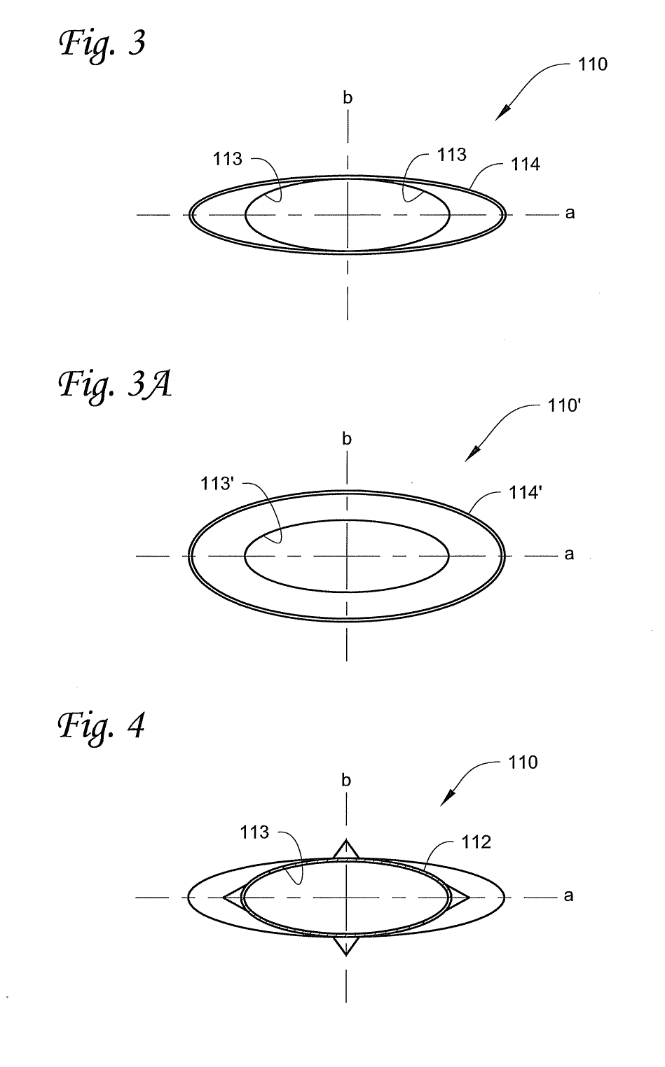

Additional features depicted in connection with the embodiment of FIG. 2 include the flared ends of the body member 110. The flared portions 115 and 117 may occupy any suitable portion of the length of the body member 110 of the stent. Although the depicted embodiment includes flared ends at both the first end 114 and the second end 116, in some embodiments only one of the ends may be flared, and in still other embodiments, neither end may be flared. FIG. 3 is an end view of the first end 114 of the stent of FIG. 2 and FIG. 4 is a cross-sectional view of the stent of FIG. 2 taken along line 4-4 in FIG. 2. The major axes (a) and the minor axes (b) are depicted in both FIGS. 3 and 4, along with reference number 113 which denotes the interior surface of the body member 110.

Although some embodiments of the stents may have non-circular cross-sectional profiles that are substantially uniform along substantially the entire length of the body member 110, the flared end or ends of the stent depicted in FIG. 2 provide some variations that may be useful in retaining the position of the stent in an esophagus.

As depicted in FIGS. 2-4, some embodiments of stent as described herein may have a first major axis (a) in a first cross-sectional profile obtained at a first location proximate the first end 114 of the body member 110 (see, e.g., FIG. 3) and a central major axis (a) in a central cross-sectional profile obtained at a central location proximate a midpoint of the length of the body member (see, e.g., FIG. 4), wherein the body member 110 is larger across the first major axis than across the central major axis. In addition, the stent may include a first minor axis (b) in the first cross-sectional profile (see, e.g., FIG. 3) and a central minor axis (b) in the central cross-sectional profile (see, e.g., FIG. 4), wherein the dimension of the body member 110 across the first minor axis is substantially equivalent to dimension of the body member across the central minor axis. In such an embodiment, the flared shape of the body member 110 may be described as being flared only in the direction of the major axis (a).

In other embodiments, however, the flared shape of the body member may flare in along both the major and minor axes (a) and (b). An end view of such an embodiment is depicted in FIG. 3A, wherein the dimensions of the stent body member 110′ are larger across both the major and minor axes (a) and (b)at the end 114′ than at a location proximate the center of the body member (as depicted by surface 113′.

The stent of FIG. 2 also includes one or more optional tissue anchors 120 located on an external surface of the body member 110. The tissue anchors 120 may be useful to resist movement of the stent in the esophagus after deployment. Although the tissue anchors 120 may be provided as discrete structures arrayed about the body member 110, in another variation, the tissue anchors may be provided in the form of a structure 130 that extends around the perimeter of the body member 110. The tissue anchors may take any number of a variety of fauns, e.g., barbs, flanges, etc.

The tissue anchors may be characterized as vertical tissue anchors that resist post-deployment movement of the body member 110 of the stent in a direction along the longitudinal axis 111 of the body member 110. In other embodiments, the tissue anchors may be characterized as rotational tissue anchors that resist post-deployment rotation of the stent about the longitudinal axis 111 of the body member 110. In still other embodiments, the tissue anchors may be constructed to resist both post-deployment movement of the stent in a direction along the longitudinal axis 111 of the body member 110 and also resist post-deployment rotation of the body member 110 of the stent about the longitudinal axis 111 of the body member 110. The tissue anchors may take any suitable shape, e.g., barbed, shark-finned, circular protrusions, flanges, etc. Examples of some potentially suitable tissue anchors may be found in, e.g., U.S. Pat. Nos. 5,591,197 (Orth et al.); 5,800,526 (Anderson et al.); 5,824,054 (Khosravi et al.); etc.

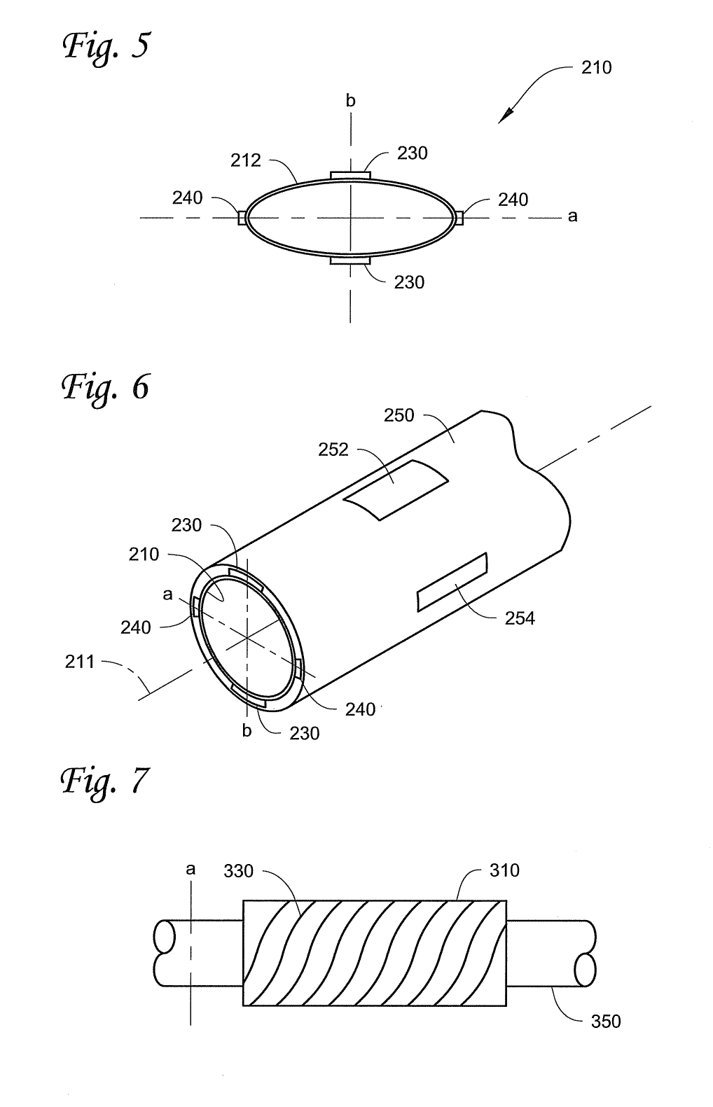

Because the non-circular stents described herein lack rotational symmetry about the longitudinal axis, accurate placement of the body members in the esophagus may be enhanced by providing indicators on the stent body member. Examples of some embodiments of indicator placement are depicted in connection with FIGS. 5 and 6. The body member 210 has a non-circular profile as described herein. The indicators 230 may be provided on the body 210 in locations that are indicative of the orientation of the minor axis (b) while the indicators 240 are provided on the body 210 in locations that are indicative of the orientation of the major axis (a).

The rotational orientation of the body member 210 about its longitudinal axis may be determined by reference to one or more of the indicators 230 and/or 240 on the wall 212 of the body member 210. Although it may be useful to provide multiple indicators, in some embodiments a single indicator may be sufficient to convey the rotational orientation of the body member 210 with respect to its longitudinal axis.

The indicators may be provided in any form that can be detected by a user. In some embodiments, the indicators may be radiopaque (such that they can be visualized using fluoroscopic imaging), they may be echogenic (such that they can be visualized using ultrasonic imaging), they may be visual (such that they can be seen by the human eye using visible light, e.g., they may be colored, they may visualized using an endoscope, etc.), etc. Also, although depicted as discrete articles, the indicators used in connection with stents as described herein may be in the form of thread or threads or any other form capable of providing the rotational orientation of the stent to a user.

FIG. 6 depicts the body member 210 of the stent restrained within a delivery device 250 that can be used to deliver the stent to, e.g., an esophagus. As seen in FIG. 6, the delivery device 250 may be in the form of a sheath such that the stent can be restrained within the interior of the delivery device 250. The need for some indication of the rotational orientation of the stent can be seen because the delivery device 250 itself may be circular in profile (or have another profile/shape that is different than the deployed non-circular profile of the stents as described herein) such that the rotational orientation of the stent 210 about the longitudinal axis 211 cannot be determined based on the shape of the delivery device 250.

In addition to (or in place of) the indicators 230 and 240 provided on the stent body member 210, the delivery device 250 may also include one or more indicators 252 and 254 that can also be used to determine the rotational orientation of the delivery device 250. If the rotational orientation of the stent within the delivery device 250 is known, then the indicators 252 and 254 can be used to provide an indication of the rotational orientation of the stent located within the delivery device 250. Cross-sectional asymmetry of the delivery device itself (i.e., an elliptical or other non-circular cross-sectional profile of the delivery device, etc.) may also serve to indicate the rotational orientation of the delivery device and, thus, the stent being delivered using the delivery device.

Although the stent of FIG. 6 is depicted as being located within the delivery device 250, in other embodiments the stents may be delivered using a delivery device that is used to carry the stent on an exterior surface. An illustrative embodiment of a delivery device 350 carrying a stent 310 on an exterior surface of the delivery device 350 is depicted in FIG. 7. An optional feature depicted in the embodiment of FIG. 7 is a constraining wire/thread 330 that may be used to indicate rotational orientation of the stent 310. The constraining wire/thread 330 depicted in FIG. 7 is wound helically, although many other variations would be possible.

Deployment or delivery of the non-circular stents described herein may be accomplished using any suitable technique or structure. In some embodiments, either the short (minor or anterior-posterior) or the long (major or left-right) axis of the stent deploys or begins to deploy first, allowing confirmation of correct stent orientation and rotational adjustment prior to full stent deployment. This may be accomplished in some embodiments by varying the length of a restraining outer sheath in the left-right vs anterior-posterior axes.

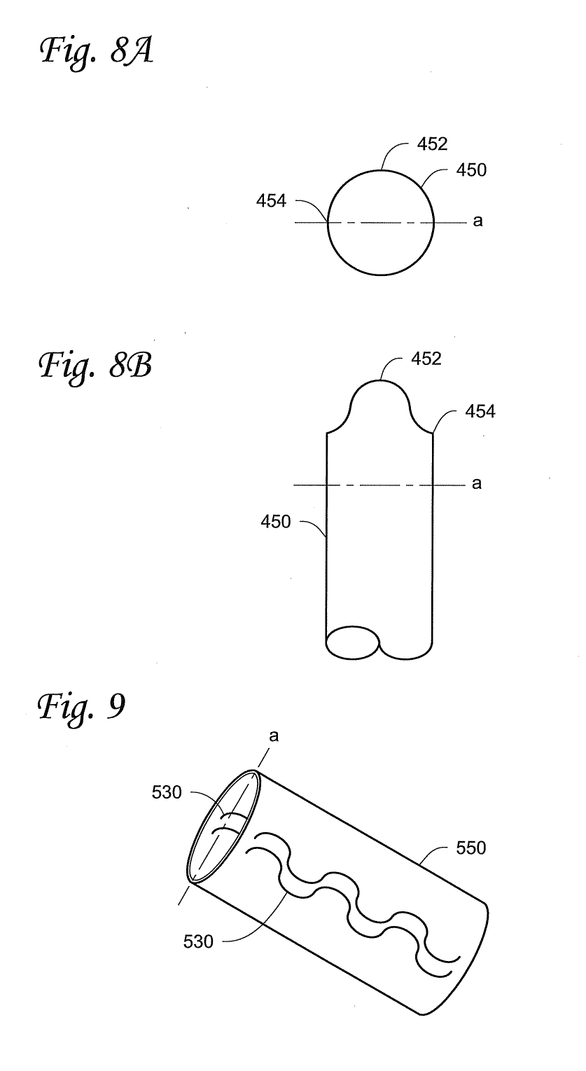

One example of such a construction is depicted in FIGS. 8A and 8B where the delivery device 450 includes extensions 452 that extend past the distal end 454 of the device 450 to denote the major sides of a non-circular stent that may be located within or on the delivery device 450. The major sides of the non-circular stent may aligned along the major axis a seen in FIGS. 8A and 8B or they may be oriented transverse to the axis a. Essentially, the extensions 452 on the delivery device 450 and a non-circular stent being delivered by the device may be arranged in any manner that would provide feedback to a user regarding the rotational orientation of the non-circular stent, e.g., aligned, transverse, etc.

Another example of a construction that may be used to indicate rotational orientation of a non-circular stent is depicted in FIG. 9 and includes articles (e.g., threads, wires, etc.) that may be visualized by any suitable manner (e.g., using the human eye, fluoroscopically, ultrasonically, etc.). The articles (e.g., threads, wires, etc.) may be arranged on the delivery device or stent in a manner that is indicative of the rotational orientation of the stent. For example, as seen in FIG. 9, the articles 530 are located proximate the center of the major sides of the non-circular device 550, where the major sides are generally aligned along the major axis a as depicted in FIG. 9.

In some embodiments, the proximal portion of a stent may deploy first.

This may be accomplished by distal movement of a restraining outer sheath of a delivery device relative to the stent itself. This distal movement may be accomplished by including a long segment of stent delivery device distal to the constrained stent itself, and/or by use of a restraining sheath that everts into or is pulled from within the central lumen of the delivery device (an example of which is described in, e.g., U.S. Patent Application Publication No. US 2008/0281398 to Koss et al.). Distal movement of the restraints on a restrained stent can also, in some embodiments, be accomplished by use of restraining threads that release the proximal portion of the stent before the more distal portions of the stent are released.

The complete disclosure of the patents, patent documents, and publications cited in the Background, the Detailed Description of Exemplary Embodiments, and elsewhere herein are incorporated by reference in their entirety as if each were individually incorporated.

Illustrative embodiments of this invention are discussed and reference has been made to possible variations within the scope of this invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. An esophageal stent comprising:
a body member comprising a first end, a second end, and a length along a longitudinal axis extending between the first end and the second end;
wherein the body member has a delivery configuration and a deployed configuration, wherein the body member is expandable from the delivery configuration to the deployed configuration;
and wherein, in the deployed configuration, the stent comprises a non-circular cross-sectional profile in the absence of any external constraints acting on the stent, wherein the cross-sectional profile is determined in a plane transverse to the longitudinal axis, and wherein the cross-sectional profile defines a major axis extending across a maximum dimension of the cross-sectional profile and a minor axis transverse to the major axis at a midpoint of the major axis, wherein the body member is larger across the major axis than across the minor axis.

2. A stent according to claim 1, wherein the non-circular cross-sectional profile is substantially uniform along substantially the entire length of the body member.

3. A stent according to claim 1, wherein the non-circular cross-sectional profile comprises an oval or elliptical cross-sectional profile.

4. A stent according to claim 1, wherein the body member comprises a first major axis in a first cross-sectional profile obtained at a first location proximate the first end of the body member and a central major axis in a central cross-sectional profile obtained at a central location proximate a midpoint of the length of the body member, wherein the body member is larger across the first major axis than across the central major axis.

5. A stent according to claim 4, wherein the body member comprises a first minor axis in the first cross-sectional profile and a central minor axis in the central cross-sectional profile, wherein the body member is larger across the first minor axis than across the central minor axis.

6. A stent according to claim 4, wherein the body member comprises a first minor axis in the first cross-sectional profile and a central minor axis in the central cross-sectional profile, wherein the dimension of the body member across the first minor axis is substantially equivalent to dimension of the body member across the central minor axis.

7. An esophageal stent comprising:
a body member comprising a first end, a second end, and a length along a longitudinal axis extending between the first end and the second end;
wherein the body member has a delivery configuration and a deployed configuration, wherein the body member is expandable from the delivery configuration to the deployed configuration;
wherein, in the deployed configuration, the stent comprises a first non-circular cross-sectional profile in the absence of any external constraints acting on the stent, wherein the first cross-sectional profile is obtained at a first location proximate the first end of the body member and is determined in a plane transverse to the longitudinal axis, and wherein the first cross-sectional profile defines a first major axis extending across a maximum dimension of the cross-sectional profile and a first minor axis transverse to the major axis at a midpoint of the first major axis, wherein the body member is larger across the first major axis than across the first minor axis
wherein the body member comprises a second major axis in a second cross-sectional profile obtained in a plane transverse to the longitudinal axis at a second location proximate the second end of the body member, wherein the second major axis extends across a maximum dimension of the second cross-sectional profile;
wherein the body member comprises a central major axis in a central cross-sectional profile obtained in a plane transverse to the longitudinal axis at a central location proximate a midpoint of the length of the body member, wherein the central major axis extends across a maximum dimension of the central cross-sectional profile;
and wherein the body member is larger across the first major axis than across the central major axis and the body member is larger across the second major axis than across the central major axis.

8. A stent according to claim 7, wherein the dimension of the body member across the first major axis is substantially equivalent to the dimension of the body member across the second major axis.

9. A stent according to claim 7, wherein the body member comprises a first minor axis in the first cross-sectional profile and a central minor axis in the central cross-sectional profile, wherein the body member is larger across the first minor axis than across the central minor axis.

10. A stent according to claim 9, wherein the body member comprises a second minor axis in the second cross-sectional profile, wherein the body member is larger across the second minor axis than across the central minor axis.

11. A stent according to claim 7, wherein the body member comprises a first minor axis in the first cross-sectional profile and a central minor axis in the central cross-sectional profile, wherein the dimension of the body member across the first minor axis is substantially equivalent to the dimension of the body member across the second minor axis.

12. A stent according to claim 11, wherein the body member comprises a second minor axis in the second cross-sectional profile, wherein the dimension of the body member across the second minor axis is substantially equivalent to the dimension of the body member across the central minor axis.

13. A stent according to claim 1, further comprising an indicator on the body member, the indicator being indicative of the orientation of the rotational orientation of the body member about the longitudinal axis.

14-15. (canceled)

16. A stent according to claim 1, the stent further comprising a tissue anchor located on an external surface of the body member.

17. A stent according to claim 16, wherein the tissue anchor comprises a vertical tissue anchor that resists post-deployment movement of the stent in a direction along the longitudinal axis of the body member.

18. A stent according to claim 16, wherein the tissue anchor comprises a rotational tissue anchor that resists post-deployment rotation of the stent about the longitudinal axis of the body member.

19. (canceled)

20. An esophageal stent delivery system comprising:
an esophageal stent that comprises:
a body member comprising a first end, a second end, and a length along a longitudinal axis extending between the first end and the second end;
wherein the body member has a delivery configuration and a deployed configuration, wherein the body member is expandable from the delivery configuration to the deployed configuration; and
wherein, in the deployed configuration, the body member comprises an oval or elliptical cross-sectional profile in the absence of any external constraints acting on the stent, wherein the cross-sectional profile is determined in a plane transverse to the longitudinal axis, and wherein the cross-sectional profile defines a major axis extending across a maximum dimension of the cross-sectional profile and a minor axis transverse to the major axis at a midpoint of the major axis, wherein the body member is larger across the major axis than across the minor axis; and
a delivery device restraining the body member in the delivery configuration.

21. A system according to claim 20, wherein the body member is restrained within an interior of the delivery device.

22. A system according to claim 20, wherein the body member is restrained on an exterior of the delivery device.

23. A system according to claim 20, wherein the delivery device comprises an indicator that is indicative of the orientation of the rotational orientation of the body member of the stent about the longitudinal axis.

24-28. (canceled)
